(19) United States
(12) Patent Application Publication
(10) Pub. No.: US 2009/0269390 A1
(43) Pub. Date: Oct. 29, 2009

(54) MEDICAL DEVICES, POLYMERS, COMPOSITIONS, AND METHODS FOR DELIVERING A HALOACETATE

(75) Inventors: Lian Leon Luo, Shoreview, MN (US); Michael Eric Benz, Ramsey, MN (US); Christopher M. Hobot, Tonka Bay, MN (US); SuPing Lyu, Maple Grove, MN (US)

Correspondence Address: MEDTRONIC, INC., 710 MEDTRONIC PARKWAY NE, MINNEAPOLIS, MN, 55432-9924, US

(73) Assignee: Medtronic, Inc., Minneapolis, MN (US)

(21) Appl. No.: 12/429,046

(22) Filed: Apr. 23, 2009

Related U.S. Application Data
(60) Provisional application No. 61/047,783, filed on Apr. 25, 2008.

(51) Int. Cl. A61L 27/54; A61K 31/19; A61K 31/215; A61P 35/00; C08F 283/02

(52) U.S. Cl. 424/426; 514/557; 514/529; 525/462

(57) ABSTRACT

Polymers, compositions, and medical devices useful for delivering (e.g., by local and/or sustained delivery) a haloacetate (e.g., dichoroacetate) to a tissue are disclosed herein. Such methods can be useful for treatment of diseases such as cancer.

This application claims the benefit of the filing date of U.S. Provisional Application No. 61/047,783, filed Apr. 25, 2008, entitled “Medical Devices, Polymers, Compositions, and Methods for Delivering a Haloacetate,” which is hereby incorporated by reference.

### BACKGROUND

Cancer is a group of diseases in which cells can grow and divide outside normal limits, invade and destroy adjacent tissues, and/or spread to other locations in the body. Many cancers are caused by abnormalities in the genetic material of transformed cells. Genetic abnormalities found in cancer cells can impact several classes of genes. For example, cancer-promoting oncogenes in cancer cells can be activated, leading to properties such as hyperactive growth and division, protection against programmed cell death, and growth and establishment in diverse tissue environments that can be outside normal tissue boundaries. In addition, tumor suppressor genes in cancer cells are often inactivated, leading to the loss of normal functions such as accurate DNA replication, control over the cell cycle, orientation and adhesion within tissues, and interaction with protective cells of the immune system.

Cancer can be treated, for example, by chemotherapy, radiation therapy, immunotherapy, monoclonal antibody therapy, surgery, or some combination thereof. Chemotherapy typically involves treatment with drugs that can destroy cancer cells. Chemotherapy drugs can interfere with cell division in various possible ways, including, for example, interfering with the duplication of DNA or the separation of newly formed chromosomes. Some forms of chemotherapy target all rapidly dividing cells, and are not specific for cancer cells. Hence, chemotherapy has the potential to harm healthy tissue.

New compositions and methods for treating cancer, that preferably reduce or eliminate the potential to harm healthy tissue, are desired.

### SUMMARY

It is widely believed that cancer cells rely primarily on non-oxidative breakdown of glucose (i.e., a process known as glycolysis) for energy production, producing pyruvate outside of the mitochondria. For many years it was widely accepted that cancer cells use inefficient glycolysis for metabolism, because irreparable damage to the mitochondria blocked the more efficient glucose oxidation pathway. More recently, the oral administration of dichloroacetate has been reported to reactivate the mitochondrial function in a cancer cell, and shift metabolism from glycolysis to glucose oxidation (e.g., Bonnet et al., Cancer Cell, 11:37-51 (2007)). The metabolism shift can also lead to the activation of apoptosis, a process by which abnormal cells self-destruct, causing cancer cells to wither and die. However, systematic oral administration of dichloroacetate (e.g., at a daily dosage about 25-50 mg/kg) has the potential to lead to undesired health consequences such as hepatotoxicity and/or neurotoxicity. As a potential remedy for at least some of the issues that may be encountered with oral delivery of dichloroacetate, medical devices, polymers, and/or compositions that can be used for local and/or sustained delivery of a haloacetate (e.g., dichloroacetate) are advantageously disclosed herein.

In one embodiment, the present disclosure provides a medical device, such as a pump or polymer that can locally deliver a haloacetate (e.g., a chloroacetate such as dichloroacetate). As used herein, the phrase “locally deliver” is intended to include a wide variety of delivery methods in which, for at least one point of time during treatment, the ratio of the concentration of the haloacetate proximate the targeted tissue divided by the concentration of the haloacetate in the blood is at least 2, and in certain embodiments at least 3, at least 4, at least 5, at least 6, at least 7, at least 8, at least 9, or at least 10. In certain embodiments, the phrase “locally deliver” is intended to exclude systemic delivery as the sole method of delivery. To locally deliver includes, for example, delivery proximate a tissue or area in need of treatment. Such tissues and areas are commonly referred to as targeted tissues and areas. As used herein, delivery “proximate” a tissue includes delivery in the tissue or sufficiently near the tissue to effect treatment of the targeted tissue. The haloacetate can be delivered, for example, via a pump, by degradation of the polymer, erosion of the polymer, and/or diffusion from the polymer. To locally deliver also includes, for example, delivery via the vasculature supplying the target tissue. For example, to treat pancreatic cancer, it might be desirable to position a catheter or a depot in an artery supplying the pancreas and deliver the drug into the artery. Local delivery also includes, for example, delivering to the target tissue through other points such as the urethra or vagina to deliver the drug. For example, a catheter is positioned in the urethra and a needle attached to the catheter goes through the urethral wall to access the prostate to treat conditions such as cancers of the prostate, bladder, uterus, cervix, colon and bone (bone could be accessed through an artery supplying it). Compositions including such polymers are also provided.

In another embodiment, the present disclosure provides a medical device, such as a pump or polymer that can provide sustained delivery of a haloacetate (e.g., a chloroacetate such as dichloroacetate). The medical device, such as a pump or polymer can be the same or different than the polymer that can locally deliver a haloacetate. As used herein, the phrase “sustained delivery” is intended to include a wide variety of delivery methods in which delivery of the haloacetate is sustained at useful levels (e.g., therapeutic levels for the desired treatment) over a sustained period of time. Useful periods of time for sustained delivery can depend on, among other things, the condition being treated. In certain embodiments, useful periods of time for sustained delivery can be greater than 1 hour, greater than 12 hours, greater than 24 hours, greater than 48 hours, greater than 72 hours, greater than one week, greater than 2 weeks, greater than 4 weeks, or greater than 6 weeks. In certain embodiments, medical devices, such as a pumps or polymers are provided that can locally deliver a haloacetate for a sustained period of time. The haloacetate can be delivered, for example, through a catheter, by degradation of the polymer, erosion of the polymer, and/or diffusion from the polymer. Compositions including such polymers are also provided.

In another embodiment, the present disclosure provides a polymer that includes at least one group of the formula (Formula I): -A-C(O)—CH₃₋ₙXₙ, wherein: A represents a heteroatom such as an oxygen atom, a nitrogen atom, or a sulfur atom; each X is independently a halogen atom (e.g. a chlorine atom); and n=1 to 3 (e.g., 2). At least one group of Formula I can be a terminal group of the polymer. In some embodiments, the polymer includes a plurality of groups of Formula I, some or all of which can optionally be attached to a repeat unit of the polymer. Compositions including such polymers are also provided.

In another embodiment, the present disclosure provides a polymer including two or more repeat units of the formula (Formula II):

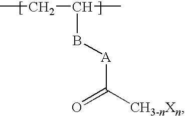

wherein: A represents a heteroatom such as an oxygen atom, a nitrogen atom, or a sulfur atom; B represents an optional linking group; each X is independently a halogen atom (e.g., a chlorine atom); and n=1 to 3 (e.g., 2). Compositions including such polymers are also provided.

In another aspect, the present disclosure provides a composition for locally delivering a haloacetate. In one embodiment, the composition includes: a polymer and a haloacetate source, which can be, for example, a haloacetate dissolved, dispersed, suspended, and/or encapsulated in the polymer. Optionally, the haloacetate source can be covalently and/or tonically attached to the polymer. The polymer can be, for example, a resorbable (e.g., bioresorbable) polymer that is optionally water soluble. Alternatively, or in addition to, the polymer can be biodegradable.

In another aspect, the present disclosure provides a composition that can provide sustained delivery of a haloacetate. The composition can be the same or different than the composition that can locally deliver a haloacetate. In one embodiment, the composition includes: a polymer and a haloacetate source, which can be, for example, a haloacetate dissolved, dispersed, suspended, and/or encapsulated in the polymer. Optionally, the haloacetate source can be covalently and/or ionically attached to the polymer. The polymer can be, for example, a resorbable (e.g., bioresorbable) polymer that is optionally water soluble. Alternatively, or in addition to, the polymer can be biodegradable.

In another aspect, medical devices including one or more of such polymers and/or compositions are also disclosed.

In another aspect, the present disclosure provides a method of preparing a polymer. The method includes combining components including at least one hydroxy-containing polymer and a haloacetate, a haloacetate ester, and/or a haloacetate anhydride under conditions effective to esterify the hydroxy-containing polymer. The haloacetate can be, for example, a haloacetic acid (e.g., dichloroacetic acid), the conjugate base of a haloacetic acid (e.g., the conjugate base of dichloroacetic acid), a salt of a haloacetic acid (e.g., a salt of dichloroacetic acid), a complex of a haloacetic acid (e.g., a complex of dichloroacetic acid), or a combination thereof. Conditions effective to esterify the hydroxyl-containing polymer can include the presence of a strong acid (e.g., trifluoroacetic acid) and/or an anhydride thereof; and/or a carbodiimide (e.g., dicyclohexylcarbodiimide).

In another aspect, the present disclosure provides a method for local delivery of a haloacetate to a tissue. The method includes locating proximate the tissue a polymer, composition, and/or medical device as disclosed herein. Locating can include injecting the polymer and/or composition proximate the tissue. In certain embodiments the method further includes degradation, erosion, and/or resorption of the polymer. Alternatively, or in addition to, such methods can also include diffusion of the haloacetate from the polymer.

In another aspect, the drug delivery device is an external or implanted drug pump system that may include a catheter coupled to the pump. In yet another aspect of the invention the drug delivery device may be a depot contained with a pump.

In another aspect, the present disclosure provides a method for sustained delivery of a haloacetate to a tissue. The method can be the same or different than the method that locally delivers a haloacetate to a tissue. The method includes locating proximate the tissue a polymer, composition, and/or medical device as disclosed herein. Locating can include injecting the polymer and/or composition proximate the tissue. In certain embodiments the method further includes degradation, erosion, and/or resorption of the polymer. Alternatively, or in addition to, such methods can also include diffusion of the haloacetate from the polymer.

In certain embodiments, medical devices, polymers, and/or compositions as disclosed herein can locally deliver and/or provide sustained delivery of therapeutic quantities of a haloacetate to treat cancer, for example, while reducing or eliminating possible undesirable, systematic side effects. In other certain embodiments, medical devices, polymers, and/or compositions used to deliver the haloacetate are resorbed by the body, thus avoiding any need for surgical removal of the polymer and/or composition. For embodiments in which a haloacetate is delivered from a device including a biodegradable polymer proximate a tissue, any need to monitor the device for potential effects to the tissue from the polymer proximate thereto are preferably reduced or eliminated by biodegradation of the polymer.

The terms “comprises” and variations thereof do not have a limiting meaning where these terms appear in the description and claims.

As used herein, “a,” “an,” “the,” “at least one,” and “one or more” are used interchangeably.

As used herein, the term “or” is generally employed in the sense as including “and/or” unless the context of the usage clearly indicates otherwise.

Also herein, the recitations of numerical ranges by endpoints include all numbers subsumed within that range (e.g., 1 to 5 includes 1, 1.5, 2, 2.75, 3, 3.80, 4, 5, etc.).

The above summary of the present invention is not intended to describe each disclosed embodiment or every implementation of the present invention. The description that follows more particularly exemplifies illustrative embodiments. In several places throughout the application, guidance is provided through lists of examples, which examples can be used in various combinations. In each instance, the recited list serves only as a representative group and should not be interpreted as an exclusive list.

### DETAILED DESCRIPTION OF ILLUSTRATIVE EMBODIMENTS

The present disclosure provides medical devices, polymers, and/or compositions that can locally deliver and/or provide sustained delivery of a haloacetate (i.e., a halogen-containing acetate). As used herein, the term “acetate” is intended to be broadly interpreted to encompass not only the anionic conjugate base (i.e., CH₃CO₂⁻) of acetic acid, but also acetic acid itself (i.e., the free acid CH₃CO₂H), salts and/or complexes of acetic acid (e.g., CH₃CO₂M and/or CH₃CO₂H.B, wherein B can represent, for example, alkyl amines including primary amines (e.g., methylamine or ethylamine), secondary amines (e.g., dimethylamine, methylethylamine, or diethylamine), tertiary amines (e.g., trimethylamine or triethylamine), and/or combinations thereof. For example, the anionic conjugate base of acetic acid can be in equilibrium with the free acid.

As used herein, the term “haloacetate” is intended to encompass monohaloacetates (i.e., including a XCH₂CO₂— group, and preferably a XCH₂CO₂— moiety), dihaloacetates (i.e., including a X₂CHCO₂— group, and preferably a X₂CHCO₂— moiety), trihaloacetates (i.e., including a X₃CCO₂— group, and preferably a X₃CCO₂— moiety), and/or combinations thereof, wherein each X independently represents a halogen atom. Consistent with the definition of “acetate” given herein above, “haloacetate” is intended to be broadly interpreted to encompass not only an anionic conjugate base (i.e., XₙCH₃₋ₙCO₂⁻) of a haloacetic acid, but also the haloacetic acid itself (i.e., the free acid XₙCH₃₋ₙCO₂H), salts and/or complexes of a haloacetic acid (e.g., XₙCH₃₋ₙCO₂M and/or XₙCH₃₋ₙCO₂H.B), and combinations thereof.

For certain embodiments of the present invention, preferred haloacetates include chloroacetates (i.e., including a ClₙCH₃₋ₙCO₂— group, and preferably a ClₙCH₃₋ₙCO₂— moiety), and a particularly preferred haloacetate is dichloroacetate (i.e., including a Cl₂CHCO₂— group, and preferably a Cl₂CHCO₂— moiety).

As used herein, the term “organic group” is used to mean a hydrocarbon group that is classified as an aliphatic group, cyclic group, or combination of aliphatic and cyclic groups (e.g., alkaryl and aralkyl groups). In the context of the present disclosure, suitable organic groups as disclosed herein are those that do not interfere with the delivery of a haloacetate as disclosed herein. In the context of the present disclosure, the term “aliphatic group” means a saturated or unsaturated linear or branched hydrocarbon group. This term is used to encompass alkyl, alkenyl, and alkynyl groups, for example. The term “alkyl group” means a saturated linear or branched monovalent hydrocarbon group including, for example, methyl, ethyl, n-propyl, isopropyl, tert-butyl, amyl, heptyl, and the like. The term “alkenyl group” means an unsaturated, linear or branched monovalent hydrocarbon group with one or more olefinically unsaturated groups (i.e., carbon-carbon double bonds), such as a vinyl group. The term “alkynyl group” means an unsaturated, linear or branched monovalent hydrocarbon group with one or more carbon-carbon triple bonds. The term “cyclic group” means a closed ring hydrocarbon group that is classified as an alicyclic group, aromatic group, or heterocyclic group. The term “alicyclic group” means a cyclic hydrocarbon group having properties resembling those of aliphatic groups. The term “aromatic group” or “aryl group” means a mono- or polynuclear aromatic hydrocarbon group. The term “heterocyclic group” means a closed ring hydrocarbon in which one or more of the atoms in the ring is an element other than carbon (e.g., nitrogen, oxygen, sulfur, etc.).

As a means of simplifying the discussion and the recitation of certain terminology used throughout this application, the terms “group” and “moiety” are used to differentiate between chemical species that allow for substitution or that may be substituted and those that do not so allow for substitution or may not be so substituted. Thus, when the term “group” is used to describe a chemical substituent, the described chemical material includes the unsubstituted group and that group with nonperoxidic O, N, S, Si, or F atoms, for example, in the chain as well as carbonyl groups or other conventional substituents. Where the term “moiety” is used to describe a chemical compound or substituent, only an unsubstituted chemical material is intended to be included. For example, the phrase “alkyl group” is intended to include not only pure open chain saturated hydrocarbon alkyl substituents, such as methyl, ethyl, propyl, tert-butyl, and the like, but also alkyl substituents bearing further substituents known in the art, such as hydroxy, alkoxy, alkylsulfonyl, halogen atoms, cyano, nitro, amino, carboxyl, etc. Thus, “alkyl group” includes ether groups, haloalkyls, nitroalkyls, carboxyalkyls, hydroxyalkyls, sulfoalkyls, etc. On the other hand, the phrase “alkyl moiety” is limited to the inclusion of only pure open chain saturated hydrocarbon alkyl substituents, such as methyl, ethyl, propyl, tert-butyl, and the like.

In one aspect, the present invention provides polymers and/or compositions including a polymer that can deliver a haloacetate upon degradation and/or erosion of the polymer. The polymer can be hydrophilic or hydrophobic. The polymer can be a thermoplastic polymer or a thermoset polymer. The polymer can be crystalline, semicrystalline, or amorphous. In certain embodiments, the polymer can include an attached group (e.g., covalently and/or ionically attached) that can deliver a haloacetate upon degradation.

The polymer can be porous or nonporous. As used herein, “porous” is used to refer to an object that has at least 50% void volume, preferably at least 55%, 60%, 65%, 70%, 75%, 80%, 85%, 90%, or even 95% or higher void volume. As used herein, “non-porous” is used to refer to an object that has less than 50% void volume, preferably at most 45%, 40%, 35%, 30%, 25%, 20%, 15%, 10%, 5%, or even 0% void volume. As used herein, “void volume” means unoccupied space, and percent void volume can be conveniently determined by dividing the density of the sample by the density of the fully-densified polymer.

Alternatively, or in addition to, the polymer can contain a haloacetate dissolved, dispersed, and/or suspended therein (e.g., encapsulated in the polymer), and the haloacetate can be delivered upon degradation and/or erosion of the polymer. As used herein, the term “degradation” is intended to be broadly interpreted to include a wide variety of reactions that break down or cleave the polymer into smaller parts. Degradation reactions may occur at one or more of the polymer backbone, pendant groups, and terminal groups. Thus, “degradation” of the polymer may or may not lead to a reduction in the chain length of the polymer. Degradation reactions are intended to include, for example, hydrolysis reactions, biodegradation reactions, enzyme catalyzed reactions such as hydrolysis, and combinations thereof. As used herein, the term “erosion” is intended to be broadly interpreted to include a wide variety of mechanisms in which the structural integrity of the polymer is diminished or eliminated. Erosion is intended to include, for example, dissolution (e.g. dissolving), resorption (e.g., bioresorption), and combinations thereof. Degradation may or may not result in erosion of the polymer, and erosion may occur with or without degradation of the polymer.

Alternatively, in one embodiment, the haloacetate and/or polymer is delivered via a pump, such as an infusion pump that administers a haloacetate or haloacetate-containing polymer through a catheter, the proximal end of which is near the predetermined target site. In other embodiments, the pump is an implantable mini-pump, an implantable controlled release device (such as, for example, the device described in U.S. Pat. No. 6,001,386, which is incorporated herein by reference), or a sustained release delivery system (such as the system described in U.S. Pat. No. 6,007,843, which is incorporated herein by reference).

An exemplary polymer that can deliver a haloacetate upon degradation has an attached group that delivers a haloacetate upon hydrolysis of the attached group. The polymer can include, for example, at least one attached group of the formula (Formula I): -A-C(O)—CH₃₋ₙXₙ, wherein: A represents a heteroatom such as an oxygen atom, a nitrogen atom, or a sulfur atom; each X is independently a halogen atom; and n =1 to 3. In certain embodiments, A represents an oxygen atom or a sulfur atom. In other certain embodiments, A represents an oxygen atom, each X is a chlorine atom, and n=2. In certain embodiments, the at least one group of Formula I can be a terminal group at one or more terminal of the polymer. In other certain embodiments in which the polymer includes a plurality of groups of Formula I, each group of Formula I can be attached, for example, to a repeat unit of the polymer.

An exemplary polymer having a group of Formula I attached a repeat unit of the polymer is illustrated as follows. The polymer can include two or more repeat units of the formula (Formula II):

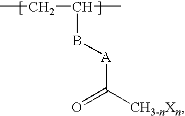

wherein: A represents a heteroatom such as an oxygen atom, a nitrogen atom, or a sulfur atom; B represents an optional linking group; each X is independently a halogen atom; and n=1 to 3. In certain embodiments, A represents an oxygen atom or a sulfur atom. In certain preferred embodiments, B is absent, A represents an oxygen atom, each X is a chlorine atom, and n=2.

For embodiments in which B is present in Formula II, B can represent a linking group such as an organic group. For some embodiments in which B is present in Formula II, B can represent a linking group such as an organic moiety (e.g., a hydrocarbon moiety). For some embodiments, B can represent a C1-C12 linking group, a C1-C8 linking group, a C1-C6 linking group, a C1-C4 linking group, a C1-C3 linking group, or a C1-C2 linking group. Exemplary divalent linking groups include, but are not limited to, methylene, 1,2-ethanediyl, 1,2-propanediyl, 1,3-propanediyl, 1,4-butanediyl, 1,6-hexanediyl, 1,8-ocatanediyl, 1,10-decanediyl, and/or 1,1 2-dodecanediyl.

In another aspect, the present invention provides compositions that include a polymer, in which the compositions can deliver a haloacetate upon degradation and/or erosion of the polymer. The polymer can be porous or nonporous. The polymer can be hydrophilic or hydrophobic. Such compositions can include one or more polymers that can deliver a haloacetate upon degradation and/or erosion of the polymer as disclosed herein above. Alternatively, or in addition to, the composition can include, for example, a resorbable polymer (e.g., a bioresorbable polymer) and a haloacetate source.

In certain embodiments, the haloacetate source can be the haloacetate itself (e.g., a free acid, a salt, and/or a complex thereof) dissolved, dispersed, suspended, and/or encapsulated in the resorbable polymer. In other certain embodiments, the haloacetate source can include, for example, one or more haloacetate esters (e.g., methyl haloacetate, ethyl haloacetate, and the like), one or more haloacetate anhydrides (e.g., dichloroacetic anhydride), and/or combinations thereof. In even other certain embodiments, the haloacetate source can be a haloacetate group that is covalently and/or ionically attached to the resorbable polymer.

Resorbable polymers include bioresorbable polymers that can undergo erosion in a tissue, followed by absorption by the body. In certain embodiments, resorbable polymers can include, for example, water soluble polymers such as polyvinyl alcohol (PVA) and polyethylene glycol (PEG); polysaccharides that are soluble in acidic media such as chitosan; and combinations thereof. In other certain embodiments, resorbable polymers can also include, for example, biodegradable polymers such as polyesters, polyorthoesters, polycarbonates, polyketals, polyamides, polyimides, polyurethanes, and combinations thereof. Exemplary resorbable polymers that are polyesters include, for example, polylactic acid (PLA), polyglycolic acid (PGA), poly(lactic-co-glycolic acid) (PLGA), polycaprolactone (PCL), and combinations thereof. An exemplary resorbable polymer that is a polycarbonate is polytrimethylene carbonate (PTMC).

In yet another aspect, the present invention provides polymers and/or compositions including a polymer, in which the polymer and/or composition can deliver a haloacetate by diffusion of the haloacetate from the polymer. The polymer can be porous or nonporous. The polymer can be hydrophilic or hydrophobic.

The polymer can be biostable or biodegradable. As used herein, “biodegradable” and “bioerodible” are used interchangeably and are intended to broadly encompass materials including, for example, those that tend to break down upon exposure to physiological environments. Biodegradable and/or bioerodible polymers known in the art include, for example, linear aliphatic polyester homopolymers (e.g., polyglycolide, polylactide, polycaprolactone, and polyhydroxybutyrate) and copolymers (e.g., poly(glycolide-co-lactide), poly(glycolide-co-caprolactone), poly(glycolide-co-trimethylenecarbonate), poly(lactic acid-co-lysine), poly(lactide-co-urethane), poly(ester-co-amide)); polyanhydrides; polyketals; and polyorthoesters.

In certain embodiments, the polymer includes an attached group (e.g., covalently and/or ionically attached) that can deliver a haloacetate upon degradation, which has been discussed herein above. Alternatively, or in addition to, the polymer can contain a haloacetate dissolved, dispersed, and/or suspended therein (e.g., encapsulated in the polymer), and the haloacetate can be delivered, for example, by diffusion of the haloacetate from the polymer. In some embodiments, diffusion of the haloacetate from the polymer occurs upon locating the polymer and/or composition proximate a tissue such that the polymer can contact bodily fluids, for example.

In another aspect, the present invention provides medical devices that include one or more polymers and/or compositions as disclosed herein above. In certain embodiments, one or more polymers and/or compositions (e.g., biostable or biodegradable) as disclosed herein can be in the form of microparticles that can function, for example, as embolic agents and/or embolic devices. In other certain embodiments, one or more polymers and/or compositions as disclosed herein can be shaped to form a medical device, preferably a biodegradable medical device. The one or more polymers and/or compositions can be shaped by methods known in the art including compression molding, injection molding, casting, extruding, milling, blow molding, or combinations thereof. As used herein, a “medical device” includes devices that have surfaces that contact tissue, bone, blood, or other bodily fluids in the course of their operation, which fluids are subsequently used in patients. In certain embodiments, medical devices can be implantable devices. In other certain embodiments, medical devices can include depots (e.g., drug depots that are implantable or non-implantable) that can, for example, store a drug, and release the drug over time. A wide variety of depots can be used such as those that can take the form of, for example, capsules, microspheres, particles, rods, gels, coatings, matrices, wafers, pills, or combinations thereof. In other certain embodiments, medical devices can be embolic devices.

Medical devices can include, for example, extracorporeal devices for use in surgery such as blood oxygenators, blood pumps, blood sensors, tubing used to carry blood, and the like which contact blood which is then returned to the patient. This can also include endoprostheses implanted in blood contact in a human or animal body such as vascular grafts, stents, pacemaker leads, heart valves, and the like, that are implanted in blood vessels or in the heart. This can also include devices for temporary intravascular use such as catheters, guide wires, and the like which are placed into the blood vessels or the heart for purposes of monitoring or repair. In certain embodiments, medical devices can include biodegradable nasal and sinus stents. In certain embodiments, medical devices can include chronically removable pacemaker leads. A medical device can also be fabricated by polymerizing components in a suitable mold.

Polymers and/or compositions as disclosed herein can also be coated onto a substrate if desired. A coating mixture of the polymer can be prepared using solvents such as toluene, chloroform, tetrahydrofuran, perfluorinated solvents, and combinations thereof. Preferred solvents include those that can be rendered moisture-free and/or those that have no active hydrogens. The coating mixture can be applied to an appropriate substrate such as uncoated or polymer coated medical wires, catheters, stents, prostheses, penile inserts, and the like, by conventional coating application methods. Such methods include, but are not limited to, dipping, spraying, wiping, painting, solvent swelling, and the like. After applying the coating solution to a substrate, the solvent is preferably allowed to evaporate from the coated substrate.

The materials of a suitable substrate include, but are not limited to, polymers, metal, glass, ceramics, composites, and multilayer laminates of these materials. The coating may be applied to metal substrates such as the stainless steel used for guide wires, stents, catheters and other devices. Organic substrates that may be coated with the polymers and/or compositions as disclosed herein include, but are not limited to, polyether-polyamide block copolymers, polyethylene terephthalate, polyetherurethane, polyesterurethane, other polyurethanes, silicone, natural rubber, rubber latex, synthetic rubbers, polyester-polyether copolymers, polycarbonates, and other organic materials.

In another aspect, the present invention provides methods of preparing polymers and/or compositions that can deliver a haloacetate upon degradation and/or erosion of the polymer. In one embodiment, the method includes combining components including at least one hydroxy-containing polymer and a haloacetate, a haloacetate ester, and/or a haloacetate anhydride under conditions effective to esterify the hydroxy-containing polymer. The haloacetate can be, for example, dichloroacetic acid, the conjugate base of dichloroacetic acid, a salt of dichloroacetic acid, a complex of dichloroacetic acid, or a combination thereof. Optionally, the components can further include a strong acid and/or an anhydride thereof. A wide variety of strong acids are well known to one of skill in the art including, for example, carboxylic acids such as trifluoroacetic acid. In certain embodiments esterification can be carried out by activating a haloacetic acid for reaction with alcohols. For example a haloacetic acid can be treated with a carbodiiimide (e.g., dicyclohexylcarbodiimide) to activate the acid for reaction with alcohols. In certain embodiments reactions can be driven to form an ester by removing water formed in the esterification reaction. For example, an esterification reaction can be carried out in a solvent that forms an azeotrope with water. In such embodiments, the reaction can be driven to form an ester by removal (e.g., azeotropic removal) of water from the reaction mixture.

A wide variety of hydroxy-containing polymers can be used in the methods disclosed herein including, but not limited to, polyurethanes (e.g., polyether urethanes, polyester urethanes including polycaprolactone urethanes), polyureas, polyurethane-ureas, polyesters (e.g., polyethylene terephthalate), poly(beta-aminoesters), polycarbonates, poly(meth)acrylates, polysulfones, polyimides, polyamides, epoxies, polyacetals, polyketals, polyorthoesters, vinyl polymers, polyanhydrides, polytriazoles, silicone rubber, natural rubber, rubber latex, synthetic rubbers, polyether-polyamide block copolymers, polyester-polyether copolymers, and combinations and/or copolymers thereof. Exemplary polyesters include, for example, linear aliphatic polyester homopolymers (e.g., polyglycolide, polylactide, polycaprolactone, and polyhydroxybutyrate) and copolymers (e.g., poly(glycolide-co-lactide), poly(glycolide-co-caprolactone), poly(glycolide-co-trimethylenecarbonate), poly(lactic acid-co-lysine), poly(lactide-co-urethane), poly(ester-co-amide)). Polymers used in the methods disclosed herein can be biostable or biodegradable.

Compositions having one or more haloacetates dissolved, dispersed, suspended, and/or encapsulated in the polymer can be prepared by a wide variety of methods known in the art. For example, such compositions can be prepared by solution processing, milling, extruding, polymerizing components in the presence of one or more haloacetates, and combinations thereof.

For certain applications, polymers and/or compositions as disclosed herein can be blended with another polymer (e.g., the same or different) to provide the desired physical and/or chemical properties. For example, two polymers having different molecular weights can be blended to optimize the delivery rate of a haloacetate. For another example, two polymers having different repeat units can be blended to provide desired physical and/or chemical properties. For even another example, a polymer of one chemical structure can be blended with a polymer of a different chemical structure to provide desired physical and/or chemical properties.

Polymers and/or compositions as disclosed herein can be used in combination with a variety of particulate materials. For example, they can be used with moisture curing ceramic materials (e.g., tricalcium phosphate) for vertebroplasty cements, bone void filling (due to disease such as cancer or due to fracture). They can be used in combination with inorganic materials such as hydroxyapatite to form pastes for use in bone healing, sealing, filling, repair, and replacement. They can be used as or in combination with polymer microspheres that can be reservoirs for a biologically active agent such as a protein, DNA plasmid, RNA plasmid, antisense agent, etc.

Alternatively, polymers and/or compositions as disclosed herein can be used in combination with other materials to form a composite (e.g., a polymer having an additive therein). In addition to the polymers and/or compositions disclosed herein, composites can include a wide variety of additives, and particularly particulate additives, such as, for example, fillers (e.g., including particulate, fiber, and/or platelet material), other polymers (e.g., polymer particulate materials such as polytetrafluoroethylene can result in higher modulus composites), imaging particulate materials (e.g., barium sulfate for visualizing material placement using, for example, fluoroscopy), biologically derived materials (e.g., bone particles, cartilage, demineralized bone matrix, platelet gel, and combinations thereof), and combinations thereof. Additives can be dissolved, dispersed, and/or suspended within the composite. For particulate additives, the additive is typically dispersed within the composite.

Polymers as disclosed herein can be combined with fibers, woven or nonwoven fabric for reconstructive surgery, such as the in situ formation of a bone plate or a bone prosthesis.

Additives that can be combined with a polymer as disclosed herein to form a composition include, but are not limited to, wetting agents for improving wettability to hydrophobic surfaces, viscosity and flow control agents to adjust the viscosity and thixotropy of the mixture to a desired level, antioxidants to improve oxidative stability of the coatings, dyes or pigments to impart color or radiopacity, and air release agents or defoamers, cure catalysts, cure accelerants, plasticizers, solvents, stabilizers (cure inhibitors, pot-life extenders), and adhesion promoters.

Optionally, the polymers and/or compositions disclosed herein can include one or more biologically active agents different than the one or more haloacetates disclosed herein. As used herein, a “biologically active agent” is intended to be broadly interpreted as any agent capable of eliciting a response in a biological system such as, for example, living cell(s), tissue(s), organ(s), and being(s). Biologically active agents can include natural and/or synthetic agents. Thus, a biologically active agent is intended to be inclusive of any substance intended for use in the diagnosis, cure, mitigation, treatment, or prevention of disease or in the enhancement of desirable physical or mental development and conditions in a subject. The term “subject” as used herein is taken to include, but is not limited to, humans, sheep, horses, cattle, pigs, dogs, cats, rats, mice, birds, reptiles, fish, insects, arachnids, protists (e.g., protozoa), and prokaryotic bacteria. Preferably, the subject is a human or other mammal.

A preferred class of biologically active agents includes drugs. As used herein, the term “drug” means any therapeutic agent. Suitable drugs include inorganic and organic drugs, without limitation, and include drugs that act on the peripheral nerves, adrenergic receptors, cholinergic receptors, nervous system, skeletal muscles, cardiovascular system, smooth muscles, blood circulatory system, synaptic sites, neuro-effector junctional sites, endocrine system, hormone systems, immunological system, reproductive system, skeletal system, autocoid systems, alimentary and excretory systems (including urological systems), histamine systems, and the like. Such conditions, as well as others, can be advantageously treated using compositions as disclosed herein.

Suitable drugs include, for example, haloacetates, polypeptides (which is used herein to encompass a polymer of L- or D-amino acids of any length including peptides, oligopeptides, proteins, enzymes, hormones, etc.), polynucleotides (which is used herein to encompass a polymer of nucleic acids of any length including oligonucleotides, single- and double-stranded DNA, single- and double-stranded RNA, DNA/RNA chimeras, etc.), saccharides (e.g., mono-, di-, poly-saccharides, and mucopolysaccharides), vitamins, viral agents, and other living material, radionuclides, and the like. Examples include antithrombogenic and anticoagulant agents such as heparin, coumadin, protamine, and hirudin; antimicrobial agents such as antibiotics; antineoplastic agents and anti-proliferative agents such as etoposide, podophylotoxin; antiplatelet agents including aspirin and dipyridamole; antimitotics (cytotoxic agents) and antimetabolites such as methotrexate, colchicine, azathioprine, vincristine, vinblastine, fluorouracil, adriamycin, and mutamycinnucleic acids; antidiabetic such as rosiglitazone maleate; and anti-inflammatory agents. Anti-inflammatory agents for use in the present invention include glucocorticoids, their salts, and derivatives thereof, such as cortisol, cortisone, fludrocortisone, Prednisone, Prednisolone, 6α-methylprednisolone, triamcinolone, betamethasone, dexamethasone, beclomethasone, aclomethasone, amcinonide, clebethasol and clocortolone.

Preferred classes of drugs include, for example, Plasmid DNA, genes, antisense oligonucleotides and other antisense agents, peptides, proteins, protein analogs, siRNA, shRNA, miRNA, ribozymes, DNAzymes and other DNA based agents, viral and non-viral vectors, liposomes, cells, stem cells, antineoplastic agents, antiproliferative agents, antithrombogenic agents, anticoagulant agents, antiplatelet agents, antibiotics, anti-inflammatory agents, antimitotic agents, immunosuppressants, growth factors, cytokines, hormones, and combinations thereof. Examples of preferred drugs are bone morphogenetic proteins (BMP) including, for example, recombinant human bone morphogenetic protein (rhBMP-2).

Suitable drugs can have a variety of uses including, but are not limited to, anticonvulsants, analgesics, antiparkinsons, antiinflammatories (e.g., ibuprofen, fenbufen, cortisone, and the like), calcium antagonists, anesthetics (e.g., benoxinate, benzocaine, procaine, and the like), antibiotics (e.g., ciprofloxacin, norfloxacin, clofoctol, and the like), antimalarials, antiparasitics, antihypertensives, antihistamines, antipyretics, alpha-adrenergic agonists, alpha-blockers, biocides, bactericides, bronchial dilators, beta-adrenergic blocking drugs, contraceptives, cardiovascular drugs, calcium channel inhibitors, depressants, diagnostics, diuretics, electrolytes, enzymes, hypnotics, hormones, hypoglycemics, hyperglycemics, muscle contractants, muscle relaxants, neoplastics, glycoproteins, nucleoproteins, lipoproteins, ophthalmics, psychic energizers, sedatives, steroids sympathomimetics, parasympathomimetics, tranquilizers, urinary tract drugs, vaccines, vaginal drugs, vitamins, collagen, hyaluronic acid, nonsteroidal anti-inflammatory drugs, angiotensin converting enzymes, polynucleotides, polypeptides, polysaccharides, and the like.

Certain preferred embodiments include a drug selected from the group consisting of podophyllotoxin, mycophenolic acid, teniposide, etoposide, trans-retinoic acids, 9-cis retinoic acid, 13-cis retinoic acid, rapamycin, a rapalog (e.g., Everolimus, ABT-578), camptothecin, irinotecan, topotecan, tacromilus, mithramycin, mitobronitol, thiotepa, treosulfan, estramusting, chlormethine, carmustine, lomustine, busultan, mephalan, chlorambucil, ifosfamide, cyclophosphamide, doxorubicin, epirubicin, aclarubicin, daunorubicin, mitosanthrone, bleomycin, cepecitabine, cytarabine, fludarabine, cladribine, gemtabine, 5-fluorouracil, mercaptopurine, tioguanine, vinblastine, vincristine, vindesine, vinorelbine, amsacrine, bexarotene, crisantaspase, decarbasine, hydrosycarbamide, pentostatin, carboplatin, cisplatin, oxiplatin, procarbazine, paclitaxel, docetaxel, epothilone A, epothilone B, epothilone D, baxiliximab, daclizumab, interferon alpha, interferon beta, maytansine, and combinations thereof.

Certain preferred embodiments include a drug selected from the group consisting of salicylic acid, fenbufen, cortisone, ibuprofen, diflunisal, sulindac, difluprednate, prednisone, medrysone, acematacin, indomethacin, meloxicam, camptothecin, benoxinate, benzocaine, procaine, ciprofloxacin, norfloxacin, clofoctol, dexamethasone, fluocinolone, ketorolac, pentoxifylline, rapamycin, ABT-578, gabapentin, baclofen, sulfasalazine, bupivacaine, sulindac, clonidine, etanercept, pegsunercept, and combinations thereof.

Compositions including a biologically active agent and a polymer as disclosed herein and can be prepared by suitable methods known in the art. For example, such compositions can be prepared by solution processing, milling, extruding, polymerizing components in the presence of a biologically active agent, and combinations thereof.

Compositions including polymers as disclosed herein (e.g., with or without a biologically active agent) can further include additional components. Examples of such additional components include fillers, dyes, pigments, inhibitors, accelerators, viscosity modifiers, wetting agents, buffering agents, stabilizers, biologically active agents, polymeric materials, excipients, and combinations thereof.

Medical devices that include one or more polymers and/or compositions as disclosed herein can have a wide variety of uses. For example, such devices can be used to locally deliver and/or provide sustained delivery of a haloacetate to a tissue by positioning at least a portion of the device including the one or more polymers proximate the tissue and allowing the one or more polymers to deliver the haloacetate, for example, through biodegradation and/or diffusion. For another example, such devices can be used to control the delivery rate of a haloacetate from a medical device, for example, by disposing a haloacetate in at least one of the one or more polymers.

The effects of the delivered haloacetates (e.g., by local and/or sustained delivery) as disclosed herein can be evaluated in vitro, for example, by using cultures or co-cultures of primary or commercially available cell lines. For example, cancer cell lines, or cells from any proliferative biopsy and/or tissue, can be utilized to evaluate the effectiveness of the delivered haloacetates as disclosed herein, and to direct the application of the methods disclosed herein.

One of skill in the art can readily determine appropriate amounts and loading levels of haloacetates and/or haloacetate sources for compositions and devices disclosed herein, depending on the therapeutic effect desired and the desired location of delivery. For example, International Patent Publication No. WO 2006/108276 (Michelakis et al.) discloses oral administration of a daily 10-100 mglkg dose (or a 25-50 mg/kg dose) of dichloroacetate for cancer treatment, with the dose optionally administered twice per day. Such doses might represent a convenient starting point for delivered doses (e.g., by local and/or sustained delivery). However, one of skill in the art might recognize that because the haloacetate can be delivered by local and/or sustained delivery, in some embodiments, lower dosages of haloacetate might exhibit acceptable therapeutic effects, without potential side effects that might be associated with higher dosages of haloacetate. Conversely, one of skill in the art might recognize that because the haloacetate is delivered by local and/or sustained delivery, in some embodiments, higher dosages of haloacetate might exhibit improved therapeutic effects, without potential side effects that might be associated with, for example, oral delivery of haloacetate.

The present invention can also provide methods for local and/or sustained delivery of a haloacetate to a tissue. In certain embodiments, the method includes locating a polymer, composition, and/or medical device as disclosed herein above, proximate the tissue. Locating the polymer and/or composition can include, for example, injecting the polymer and/or composition proximate the tissue via a needle or catheter. In some embodiments, no additives would be needed to form an injectable composition. In some embodiments, one or more polymers can be combined with a solvent such as N-methyl-2-pyrrolidone or dimethylsulfoxide (DMSO), which are fairly biocompatible solvents. The solvent can diffuse away after injection and the polymer can remain in place. Such injectable materials can be applied proximate a desired site (e.g., a surgical site) using a syringe, catheter, applicator, or by hand.

Also, injectable compositions could include crosslinkers (such as diacrylates), plasticizers (such as triethyl citrate), lipids (soybean oil), poly(ethylene glycol) (including those with the ends blocked with methyls or similar groups), silicone oil, partially or fully fluorinated hydrocarbons, N-methyl-2-pyrrolidone, or mixtures thereof.

In certain embodiments, methods for local and/or sustained delivery of a haloacetate to a tissue can further include, for example, hydrolysis and/or resorption of the polymer. In other certain embodiments, the polymer can deliver a haloacetate by a variety of mechanisms including, for example, delivery from pores in the polymer, diffusion from the polymer, delivery through degradation of the polymer, or combinations thereof.

Other embodiments include delivery of the polymer and/or composition as disclosed herein above in semisolid and/or solid formulations designed to provide continuous and/or controlled delivery of the haloacetate into the tissue-biomaterial interface or surrounding tissue. In certain embodiments, the polymer and/or composition as disclosed herein above can be delivered into the extracellular space from which it can be diffused, distributed, contacted with, andlor internalized by cells and/or tissue. In another embodiment, the polymer and/or composition as disclosed herein above can be delivered as a component of a hydrogel that solidifies upon contact with living tissue for delivery to targeted cells and/or tissues. In yet another embodiment, the polymer and/or composition as disclosed herein above can be delivered in a solid form (e.g., films, pellets, microspheres, and the like), for delivery to targeted cells and/or tissues as biodegradation occurs. Other applications include, for example, limiting tumor growth.

The present invention is illustrated by the following examples. It is to be understood that the particular examples, materials, amounts, and procedures are to be interpreted broadly in accordance with the scope and spirit of the invention as set forth herein.

### EXAMPLES

All parts, percentages, ratios, and the like in the examples are by weight, unless noted otherwise. Mn represents number average molecular weight, and M, represents weight average molecular weight. Solvents and other reagents used were obtained from Sigma-Aldrich Chemical Company; Milwaukee, Wis. unless otherwise noted.

### Example 1

### Preparation of Poly(vinyl dichloroacetate) from Poly(vinyl Alcohol)

Poly(vinyl dichloroacetate) was prepared following a procedure similar to that described by Morgan, J. Amer. Chem. Soc., 73(2): 860-861 (1951). Dichloroacetic acid (18 mL) and trifluoroacetic anhydride (1 mL) were mixed in a dry round bottom flask. While stirring magnetically, polyvinyl alcohol (5.0 grams, average Mₙ=13,000-23,000 g/mol) was added. The flask was heated slowly to 90° C. and the mixture was stirred overnight (approximately 16 hours). The resulting viscous mixture was diluted with acetone and quenched with pentane. The material was further purified by dissolution in acetone and precipitation with pentane. Drying under vacuum gave a flaky brown solid, 6.45 grams, which was freely soluble in tetrahydrofuran. The material had Mₙ=52,500 gram/mol and polydispersity index (PDI)=1.38 by gel permeation chromatographic (GPC) analysis.

### Example 2

### Preparation of Polylactic Acid with a Dichloroacetate (DCA) End Group

In a dry round bottom flask, polylactic acid (15.0 grams, intrinsic viscosity=5.71) and dichloroacetic acid (4.5 grams) were warmed slowly to 135° C. under nitrogen. The material melted and eventually stirred freely with a magnetic stir bar. The material was purified via dissolution in tetrahydrofuran and precipitation with heptane, followed by removal of solvent under vacuum at room temperature. The material had Mₙ=2100 and polydispersity index (PDI)=1.52 by gel permeation chromatographic analysis. ¹H-NMR (acetonitrile-d₃, 300 MHz): 1.5 parts per million (ppm; multiplet, CH₃CH from lactic acid unit), 5.05 and 5.2 ppm (multiplet, CH₃CH from lactic acid unit), 6.3 ppm (singlet, CHCl₂CO). The molar ratio of the signals from dichloroacetate to lactic acid unit was approximately 1:30. The end group analysis and the GPC data suggested there was approximately one dichloroacetate functionality in each polymer chain.

### Example 3

### Synthesis of Polytrimethylene Carbonate with a Dichloroacetate (DCA) End Group

In a dry round bottom flask, trimethylene carbonate (20.5 grams) and dichloroacetic acid were heated to 1 30° C under nitrogen overnight (approximately 16 hours). The resulting brownish semi-solid was dissolved in toluene/tetrahydrofuran and separated by adding methanol.

The material that separated was dried under vacuum. The material had Mₙ=4000 gram/mol and polydispersity index (PDI)=1.6 by gel permeation chromatographic analysis. ¹H-NMR (acetonitrile-d₃, 300 MHz): 1.95 ppm (multiplet, OCH₂CH₂CH₂O from trimethylene carbonate unit), 4.15 (multiplet, OCH₂CH₂CH₂O from trimethylene carbonate unit), 6.25 ppm (singlet, CHCl₂CO). The molar ratio of the signals from dichloroacetate to trimethylene carbonate unit was approximately 1:40. The end group analysis and the GPC data suggested there was approximately one dichloroacetate functionality in each polymer chain.

### Example 4

### (Prophetic) Esterification of Dichloroacetic Acid with Sucrose

Sucrose, a water soluble carbohydrate, is reacted with dichloroacetic acid to form a hydrophobic material. The reaction is carried out with dicyclohexylcarbodiimide to catalyze the formation of ester bonds. The hydrophobic material can release a dichloroacetate upon hydrolysis of a labile ester linkage between the dichloroacetate functionality and the carbohydrate. The final hydrolysis products are water soluble carbohydrate and a dichloroacetate.

### Example 5

### (Prophetic) Incorporation of a Dichloroacetate in a Polyorthoester

Dichloroacetic acid is reacted with pentaerythritol, a compound having a plurality of hydroxy groups. The number of unreacted hydroxy groups is controlled by adjusting the stoichiometry of the starting materials. The resulting alcohol is reacted with the ketene acetal 3,9-diethylidene-2,4,8,10-tetraoxaspiro[5,5]-undecane) (DETOSU) to form a polyorthoester. The polyorthoester releases a dichloroacetate upon, for example, biodegradation.

### Example 6

### (Prophetic) Preparation of Microspheres Containing a Dichloroacetate

Microspheres containing a dichloroacetate (e.g., ethyl dichloroacetate) are prepared using a polylactic acid polymer (e.g., poly (D,L-lactide, Mₙ=50 to 100 kg/mol). The microspheres are prepared, for example, by spray drying or by drying an emulsion or dispersion. The loading of the dichloroacetate, the size of the microspheres, and the specific polymer used are independently varied to control the release profile of the dichloroacetate.

### Example 7

### (Prophetic) Preparation of a Rod from a Dichloroacetate-Containing Polymer

A mixture of a polymer (e.g., poly (D,L-lactide)) and a dichloroacetate (e.g., ethyl dichloroacetate) is heated to soften (e.g., 160° C.) and extruded through a cylindrical die to prepare a rod. The loading of the dichloroacetate, the shape of the rod, and the specific polymer used are independently varied to control the release profile of the dichloroacetate.

### Example 8

### (Prophetic) Preparation of a Coating from a Dichloroacetate-Containing Polymer

A mixture of a polymer (e.g., poly (D,L-lactide)) and a dichloroacetate (e.g., ethyl dichloroacetate) is coated on a medical device (e.g. stent). The polymer and dichloroacetate are coated, for example, by spray coating, dip coating, or a combination thereof. The loading of the dichloroacetate, the thickness of the coating, and the specific polymer used are independently varied to control the release profile of the dichloroacetate.

The complete disclosure of all patents, patent applications, and publications, and electronically available material cited herein are incorporated by reference. The foregoing detailed description and examples have been given for clarity of understanding only. No unnecessary limitations are to be understood therefrom. The invention is not limited to the exact details shown and described, for variations obvious to one skilled in the art will be included within the invention defined by the claims.

What is claimed is:

1. A polymer adapted to locally deliver a haloacetate in a subject.

2. The polymer of claim 1, wherein the local delivery is sustained delivery.

3. The polymer of claim 1, wherein the haloacetate is a chloroacetate.

4. The polymer of claim 3 wherein the chloroacetate is dichloroacetate.

5. The polymer of claim 1, wherein the polymer is adapted to deliver the haloacetate upon degradation and/or erosion of the polymer.

6. The polymer of claim 1, wherein the polymer is in the form of microparticles.

7. A polymer comprising at least one group of the formula (Formula I):
-A-C(O)—CH₃₋ₙXₙ,
wherein:
A represents a heteroatom;
each X is independently a halogen atom; and
n=1 to 3.

8. The polymer of claim 7, wherein A represents an oxygen atom, each X is a chlorine atom, and n=2.

9. The polymer of claim 7, wherein the at least one group of Formula I is a terminal group of the polymer.

10. The polymer of claim 7, wherein the polymer comprises a plurality of groups of Formula I.

11. The polymer of claim 7, wherein the polymer is in the form of microparticles.

12. The polymer of claim 7, comprising two or more repeat units of the formula (Formula II): 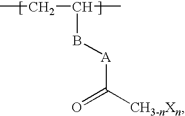
wherein:
A represents a heteroatom;
B represents an optional linking group;
each X is independently a halogen atom; and
n=1 to 3.

13. The polymer of claim 12 wherein A represents an oxygen atom, each X is a chlorine atom, and n=2.

14. A composition for delivering a haloacetate, the composition comprising:
a polymer; and
a haloacetate source.

15. The composition of claim 14, wherein the composition is adapted to locally deliver a haloacetate.

16. The composition of claim 14, wherein the composition is adapted to provide sustained delivery of a haloacetate.

17. The composition of claim 14, wherein the haloacetate source comprises a haloacetate dissolved, dispersed, suspended, and/or encapsulated in the polymer.

18. The composition of claim 14, wherein the haloacetate source is covalently and/or ionically attached to the polymer.

19. The composition of claim 14, wherein the polymer is selected from the group consisting of polyvinyl alcohol (PVA), polyethylene glycol (PEG), and combinations thereof.

20. The composition of claim 14, wherein the polymer is selected from the group consisting of polyesters, polyorthoesters, polycarbonates, polyketals, polyamides, polyimides, polyurethanes, and combinations thereof.

21. The composition of claim 20, wherein the polymer is a polyester selected from the group consisting of polylactic acid (PLA), polyglycolic acid (PGA), poly(lactic-coglycolic acid) (PLGA), polycaprolactone (PCL), and combinations thereof.

22. The composition of claim 20, wherein the polymer is a polycarbonate and the polycarbonate is polytrimethylene carbonate (PTMC).

23. An implantable medical device comprising a composition according to claim 14.

24. The medical device of claim 23, wherein the device is a depot.

25. The medical device of any one of claims 23, wherein the device is an embolic device.

26. A method of preparing a polymer, the method comprising combining components comprising at least one hydroxy-containing polymer and a haloacetate, a haloacetate ester, and/or a haloacetate anhydride under conditions effective to esterify the hydroxy-containing polymer.

27. A method for local delivery of a haloacetate to a tissue, the method comprising locating a polymer according to claim 1 proximate the tissue.
